(19) United States
(12) Patent Application Publication
(10) Pub. No.: US 2012/0128753 A1
(43) Pub. Date: May 24, 2012

(54) MEDITOWEL PRE-PACKAGED MEDICATED MUSCLE AND JOINT PAIN RELIEF WIPE

(76) Inventor: Sally Villalobos, Montebello, CA (US)

(21) Appl. No.: 12/948,780

(22) Filed: Nov. 18, 2010

(51) Int. Cl. A61K 9/70; A61K 36/81; A61P 29/00; A61K 31/60; A61P 25/00; A61K 31/05; A61K 31/125

(52) U.S. Cl. 424/443; 514/729; 424/760; 514/692; 514/159

(57) ABSTRACT

Single use, substrate uniformly impregnated with an anhydrous muscle and joint pain relief solution for the use of aseptically delivering a single dose of a muscle and joint pain relief solution to the area of pain. Solution and substrate is contained encapsulated within a heat sealed foil enclosure that is stable for at least five years.

### CROSS-REFERENCE TO RELATED APPLICATION

### STATEMENT REGARDING FEDERALLY SPONSORED RESEARCH OR DEVELOPMENT

Not applicable

### THE NAMES OT THE PARTIES TO A JOINT RESEARCH AGREEMENT

Not applicable

### INCORPORATION-BY-REFERENCE OF MATERIAL SUBMITTED ON A COMPACT DISC

Not applicable

### BACKGROUND OF THE INVENTION

1. Field of the Invention

The invention relates to a medicated muscle and joint pain relief wipe. More particularly, it relates to a pre-packaged single use disposable towelette/wipe that contains a full dose of a muscle and joint pain relief over-the-counter medication in liquid form.

2. Description of Related Art Including Information Disclosed Under 37 CFR 1.97

Muscle and Joint pain relief medication can be readily found throughout the country from grocery stores to pharmacies. These products come in all forms, from sprays to lotion to even gels. These products are packaged in many types of bottles and sizes, but none provide the portability and effectiveness of our invention.

First, a general composition for joint relief can be found in a number of U.S. patents: U.S. Pat. No. 7,282,224 to Roederer (Pain Relief Composition); U.S. Pat. No. 5,962,532 to Campbell et al. (Therapeutic method with capsaicin and capsaicin analogues); this invention builds upon these prior patents and a novel and unique product is created that provides portable muscle and joint pain relief.

First and foremost, the application is for an individually wrapped muscle and join pain relief wipe. The use of the product as a portable wipe differs from other pads and products. Instead of a pad that must be attached via adhesive to the area of pain, this product coats the area with a single dose of the muscle and joint pain relief composition at an over-the-counter (“OTC”) level. Each wipe is individually enclosed in an impermeable package that can maintain the wipes potency for at least 5 years. This allows the user to quickly administer the medication anywhere without having the nuisance of a bulky adhesive pad.

The novel concept of the muscle and joint pain relief wipe has rooted in the proprietary medicated composition. The main compositions consist of Menthol, Capsicum, Methyl Salicylate and Camphor. While other additives are generally used for cosmetics purposes, those can include: Menthol, Propylene Glycol, SD Alcohol, Glycerin, Sorbitol, and even Aloe Vera Gel.

Capsicum has also been used in compositions for muscle and joint pain relief for a number of years. U.S. Pat. No. 4,592,912 to Nickolaus (Ache and pain relieving and preventing composition); U.S. Pat. No. 5,958,418 to Prillerman (External herbal composition for treating muscle ache and joint pain).

Another important component to the composition is the use of Methyl Salicylate. Methyl Salicylate is found in a number of pain relief patents. U.S. Pat. No. 6,528,076 to Small (Topical compositions and methods for treating pain); U.S. Pat. No. 5,853,768 to Altadonna (Topical preparation and method for pain relief). The use in Methyl Salicylate in treating pain is also present in this application.

Camphor is also found in the “768” patent mentioned above (Topical preparation and method for pain relief) as well as U.S. Pat. No. 6,444,238 to Weise (Pain relief composition and method of relieving pain).

Methylparaben is also mentioned in the '076 patent (Topical composition and methods for treating pain). While it is not mentioned in the claims, it is a composition discussed in a pain relief patent.

The remaining additives are important but not essential and used for cosmetic purposes.

The Muscle and Joint Pain Relief towelette is enclosed in an impermeable package that can maintain the wipes potency for at least 5 years.

The following listed patents further illustrate less relevant pre-packaged sheet materials saturated with medicaments: U.S. Pat. No. 2,402,982 to Steenbergen (bandage in petrolatum): U.S. Pat. No. 2,999,265 to Duane et al. (hermetically sealed pad impregnated with epidermal bacteriostatic agent, humectant and emollient): U.S. Pat. No. 3,783,869 to Schnipper (medicated sanitary napkin).

Although a patent have been granted for pain relief compositions using similar and some of the same components, the utility of having an aseptic pre-packaged portable wipe that can be used to apply the topical composition is novel.

### BRIEF SUMMARY OF THE INVENTION

The present invention is focused on a pre-packaged muscle and joint pain relief towelette/wipe. The wipe is manufactured as a substrate uniformly impregnated with medicated anhydrous solution. Stability studies show that the substrate, which as a product, is enclosed in a package, will hold the wipes potency and moisture for more than 5 years. In use, the towelette/wipe is a portable single use towelette that provides the user with a single dose of the muscle and joint pain relief solution that stops muscle and joint pain in situations where portability is necessary. The towelette is wiped over the area of the skin where muscle or joint pain is detected. Using the towelette facilitates delivery of and the medicated composition to massage and penetrate the skin providing instant or near instant pain relief. After treatment the wipe is discarded.

### BRIEF DESCRIPTION OF THE DRAWINGS

FIG. 1 is a perspective view of the package enclosing the wipes

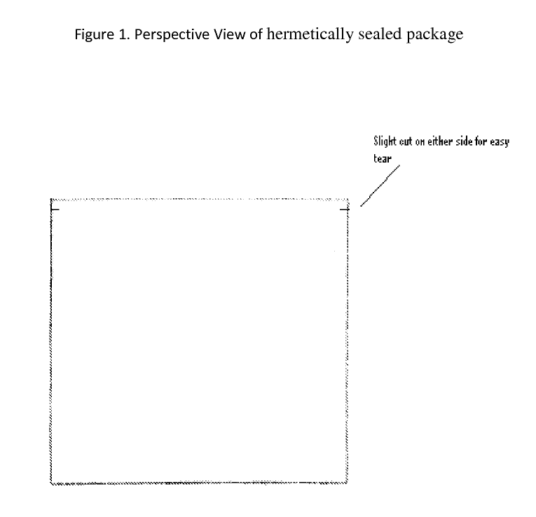

### DETAILED DESCRIPTION OF THE INVENTION

Muscle and Joint pain relief products have been in the market for some time. In the last two decades, products such as muscle and joint pain relief gels, sprays, and cream are commonly found in grocery markets, pharmacies and sporting goods stores. More recently, muscle pain relief patches have had a prevalent sighting in the aisle occupied by the mentioned products. Items not found on those aisles are portable palm size muscle and joint pain relief wipes.

These wipes have all the benefits of portability without the downside of bottles and sprays. The muscle and joint pain relief cream or gel can dehydrate and/or become contaminated and lose its potency over time. When a bottle is used, it is exposed to air and bacteria. When the user approaches the end of a bottle, the bottle has been used and exposed countless times. In applying the medicine the user must “eyeball” the amount to apply onto the area of pain, thereby applying either too much or little of the medicine. Sprays also approach the same problems. A user may spray less than the recommended dose or more than is needed. The cans must also be kept at proper temperatures ranging from 32° F. to 120° F., which prevent storage and use problems in regards to portability and eco friendly waste.

The practicality of this invention is quite simple. Instead of using a spray bottle, tube or jar of cream which is quite large, not as portable and does not maintain potency, this invention packages the wipes in a thin enclosed package this is portable, travel friendly, airport safe and stable for at least 5 years. The substrate used in the present invention comprises synthetic fiber in a non-woven, woven of knitted form. Of particular interest for use in the substrate employed in the present invention are the following fibers: polypropylene, polyester and rayon.

The pain relief composition consists of a number of components found in patented pain relief solution. The components that make up the composition currently in the marketplace includes: Menthol, Capsicum, Methyl Salicylate, Camphor, Arnica Extract, Aloe Vera gel, Sorbitol, Glycerin, SD Alcohol, Propylene Glycol, Methylparaben and Fragrance. While a majority of the components for pain relief are commonly found, the additional components are important for the solubility, consistency of the solution and ability to bind and preserve the substrate.

Menthol is the main active ingredient in MediTowels Muscle and Joint Pain Relief. Menthol is an organic compound that can either be made synthetically or obtained from peppermint or other mint oils. It is a waxy, crystalline substance, clear or white in color, which is solid at room temperature and melts slightly above. The main form of menthol occurring in nature is (−)-menthol, which is assigned the configuration of (1R,2S,5R). Menthol has local anesthetic properties Menthol's ability to chemically trigger the cold-sensitive TRPM8 receptors in the skin and is responsible for the well-known cooling sensation that it provokes when inhaled or applied to the skin.

Capsicum originates in nature from the chili pepper, specifically from the Solanaceae family. The fruit of the capsicum plant contains a chemical called capsaicin. Capsaicin reduces pain sensations when applied to the skin. Used externally as a pack or liniment, it stimulates circulation, and increases the flow of nutrients to the tissues. Scientific studies show that it is effective in pain relieve in muscles, muscle spasms, bursitis and nerve pain in people with diabetes when applied to the skin.

Methyl Salicylate commonly known as wintergreen, Methyl Salicylate is also found in nature originating in plants. Among its many uses Methyl salicylate is used as a rubefacient in deep heating, penetrating that heat to muscles and joints. Menthol has analgesic properties that are mediated through a selective activation of k-opioid receptors. In this sense it is similar to capsaicin where it stimulates heat sensors without causing an actual change in temperature.

Camphor is abundant in the oils of coniferous trees and has been used for centuries as an aromatic ingredient. Camphor is readily absorbed through the skin and produces a feeling of cooling similar to that of menthol and acts as slight local anesthetic aiding in the pain relief process.

The use of propylene glycol instead of water as a solvent is essential. Propylene glycol does not precipitate the release of free iodine, and thereby depletes its effectiveness before its actual use. Propylene glycol, unlike water, actually does preserve the stability of the active and inactive ingredients and facilitates an extended shelf life of the treated towelette/wipe also known as substrate. Propylene glycol is a lubricious emollient imparting soothing and softening qualities of the skin. Propylene glycol is considered one of the most common moisture-carrying vehicle in cosmetic and OTC drug products second only to water. Propylene glycol does not freeze in cold weather reinforcing the stability of the overall product and negates the formation of any crystallization. The use of propylene glycol, as a non-aqueous solvent obviates the need for buffers, stabilizers, and preservatives which are generally required to be used in aqueous solutions.

A small amount of Alcohol not only has antimicrobial properties but also aids to solubilize the menthol.

Glycerin aids in moisturizing and coating the injured skin keeping it hydrated while it heals. Glycerin further helps to restore the healthy look and feel to the skin without introducing water which may dehydrate too quickly and not give the pain healing ingredients time to penetrate.

Aloe Vera for centuries has been known for its natural skin healing properties. It is known for naturally soothing burns, promoting healing and cellular renewal.

Arnica extract is traditionally known for its effect on reducing bruising of impacted skin. Arnica has been demonstrated to act as an anti-inflammatory and assist normal healing processes by facilitating transport of blood and fluid accumulations through a dilating action of subcutaneous blood capillaries thus aiding in reducing inflammation in muscles and joints.

Methylparaben and Propylparabens aid in preserving the substrate.

The package of the wetted wipe is a laminate film that creates a barrier vehicle whose properties not only contains the medicated solution and substrate but prevents evaporation and contamination. Suitable and stable barrier materials are laminates, where a layer of ethylene vinyl alcohol or propylene vinyl alcohol or aluminum is sandwiched between polymeric film layers, such as low density polyethylene. Further suitable barrier materials for forming the package for the cleaning wipe herein are metallized, particularly aluminized plastics films; metal foils; oriented polyethylene terephtalate (PET); glycol modified PET; oriented polyamidel; or polymeric films. Basically every material known in the art for this purpose is suitable for use herein. A very suitable laminate material for this purpose is for instance disclosed in EP 696,991.

The package of the wipes comprises opening aids for facilitating opening of the package. Exemplary opening aids are tear tapes, cuts or the like.

What is claimed is:

1. A packaged muscle and joint pain relief wipe, comprising of
A disposable, flexible substrate suitable in size and strength for single-use application of a muscle and joint pain relief medication solution; and
A muscle and joint pain relief solution impregnated in said disposable, absorbent sheet material for providing over the counter strength muscle and joint pain relief to said sheet material; and,
A hermetically sealed envelope retaining said disposable, absorbent sheet-material.

2. A packaged muscle and joint pain relief wipe according to claim 1, wherein said hermetically sealed package is a tear-open packet.

3. An muscle and joint pain relief solution according to claim 1, wherein the muscle and joint pain relief composition consist of Menthol, Capsicum, Camphor, and Methyl Salicylates to provide muscle and joint pain relief.

4. An muscle and joint pain relief solution according to claim 1, wherein the solution also contains a combination of Methylparaben, propylene glycol, glycerin and other additives to help with solubility, consistency of the solution and ability to bind and preserve the substrate.
